(19) United States
(12) Patent Application Publication
(10) Pub. No.: US 2009/0258846 A1
(43) Pub. Date: Oct. 15, 2009

(54) Eyebrow re-growth stimulator for re-growth of lost eyebrows

(75) Inventor: Linda Sue Apple, (US)

Correspondence Address: Linda Sue Apple, 922 West Hospital Rd, Paoli, IN, 47454, US

(73) Assignee: Linda Apple

(21) Appl. No.: 12/082,269

(22) Filed: Apr. 11, 2008

(51) Int. Cl. A61K 31/506; A61K 31/56; A61Q 7/00

(52) U.S. Cl. 514/171; 514/275

(57) ABSTRACT

A chemical composition and method of an eyebrow re-growth stimulator for women who has lost there eyebrows due to dease and treatments. The lose of harmons and other causes such as in ageing women population. The eyebrow regrowth composition methods of topical treatment in an effective amount of the treatment which emploes a vasodilator in combination with estradiol and/or a 5-a-reductase inhibitor in a pharmaceutically acceptable vehicle. The compositions and methods of the invention are suitable for the treatment of lost eyebrows whereby the promotion of re-growth is increased.

Some woman lose eyebrows because of thyroid disease in addition, hormone changes, age, and vary other reasons, which can be devastation to a woman. The conventional methods of use of eyebrow pencil powders are not the most natural looking or easy to apply without actual eyebrows to pattern by. The eyebrow stimulator allows the re-growth of eyebrows making it easer to apply in a more of a natural pattern for the brows to be colored with with brow colors the stimulator provides re-growth from the inactive hair follicle by the chemical composition of minoxidil in addition, mixtures thereof.

### U.S.A. PATENT REFERENCES

application Ser. No. 06/181,959, Jun. 24, 1986

application Ser. No. 11/827,499, Jul. 12, 2007

application Ser. No. 06/659,121, Oct. 9, 1984

application Ser. No. 07/283,646, Dec. 13, 1998

application Ser. No. 07/088,767, Aug. 16, 1988

application Ser. No. 08/837,190, Apr. 14, 1997

application Ser. No. 07/218,702, Jul. 13, 1998

application Ser. No. 08/376,468, Nov. 26, 1996

application Ser. No. 07/134,422, May 14, 1991

### STATEMENT REGARDING FEDERALLY SPONSORED RESEARCH DEVELOPMENT

“Not applicable”

### REFERENCE TO SEQUENCE LISTING A TABLE OR COMPUTER PROGRAM LISTING COMPACT DISK APPENDIX

“Not appeasable”

### BACKGROUND OF INVENTION

The problems that have been presented to me over thirty years of women asking what to about the loss of their eyebrows, and what to do about them. Not until I had the problem and started to use Rogain on my own eyebrows for one year did I realize the need that the rest of the women of the population can benefit from the use of a chemical composition to stimulate eyebrow re-growth, which I did some research on this matter. The use of minoxidil has been since used to promote new hair growth, most commonly by topical application.

Minoxidil's vasodilator effects on the eyebrows is one of monoxidil proposed mechanisms to promote hair growth. It is clear that that the identification of women benefiting from the treatment a chemical composition in the form of such a solution, would be desirable to develop a treatment for the loss of eyebrows by promoting eyebrow re-growth.

### BRIEF SUMMERY OF THE INVENTION

The present invention provides a woman the opportunity to have eyebrow growth provided by chemical composition and method for promoting hair growth.

The methods of the present invention generally comprises of the treatment for patients in need of eyebrows, with the therapeutically effective amount of a vasodilator in a chemical combination with either an estradiol; or a 5-a-reducatase inhibitor of a mixture of all three, preferably sufficient to promote hair growth for eyebrows. The method preferably include administration of the selected agents from a pharmaceutically acceptable vehicle; however, the agents can be concomitantly administered in a combination of topical application.

The chemical composition of the of a comprise mixtures of these agents, a vasodilator plus an estradiol of 5-a-reductase inhibitor; or a mixture of all three preferably in a topical dosage.

### BRIEF DESCRIPTION OF SEVERAL VIEWS OF THE DRAWINGS

“Not applicable”

### DETAIL DESCRIPTION OF THE INVENTION

The present invention relates to a chemical composition and method for promoting eyebrow re-growth. In accordance with the chemical composition in a therapeutically effective amount of mixture agents, comprising a vasodilator in combination with an estradiol and/or a 5-a-reducatase inhibitor, is formulated and administered to a patient present invention, to promote eyebrow growth. The method of the invention finds use in many patients seeking maintain or increase eyebrow growth.

It is believed by theory that local tissue hypoxia may be the underlying path physiology by thyroid diseases, hormones, age, and androgens interact to cause the loss of eyebrows. The method of the chemical composition is based on the recognition of underlying path physiology. That while this chemical composition treatment is used by topical administration, nothing herein should be construed to limit the mode of delivery. It should be appreciated that each agent of the chemical combination need be administered in topical treatment preferably administered concomitantly in a highly preferred embodiment of the selected agents are administered from a single vehicle in unit dosage forms a topical transdermal treatment.

It will be appreciated that the actives useful in the chemical composition and may also employed in pharmaceutically acceptable such as esters, salts.

In the chemical composition the amount of vasodilator, estradiol and 5-a-reductase inhibitor to be administered will vary from person to person. Generally, a therapeutically effective amount of the chemical composition of the compound will be applied for a therapeutically effective duration, by therapeutically effective amount and therapeutically effective duration is preferably meant an amount or duration effective to achieve desired results.

Without limitation, it is contemplated that the vasodilators useful in the chemical composition may operate by any mechanism of action and may preferably be selected from the group consisting of minoxidil, nitroglycerin, diazoxide, nifedrpine, and mixtures of cyproterone acetate DHT exerts its effects by blocking the binding of DHT and digydrotestostrone to its reactors and mixtures thereof. Calcium ion influx inhibitors calcium channel blockers such as nifedipine, are also contemplated as preferred vasodilators in the chemical composition.

Another highly preferred vasodilator for use in the chemical compositions and method is nitroglycerin, available from various companies in various forms. Nintr-Dur by Key Pharmaceuticals, Inc. in the chemical composition.

A highly preferred estradiol for use in the compositions and methods of the chemical composition include 17 B-estradiol, marketed by Mead Johnson Laboratories under the trademark Estrace. Esterdiol is believed to be the most potent physiologic estrogen and is the major endogenous estrogenic hormone. 17 B-estradiol is chemically described as estra-1,3,5(10)-trine-3,17 B-diol in liquid form.

Another highly preferred estradiol is an estradiol transdermal system marketed by CIBA Pharmaceutical Company under the registered trademark Estraderm. Esterderm is designed to release 17 B-esterdiol through a rate limiting membrane continuously upon application to intact skin. Esterderm is available in two dosage systems, to provide nominal in vivo delivery of 0.05 mg of 0.1 mg of estradiol per day via skin of average permeability estradiol USP and 0.3 ml of alcohol USP.

A highly preferred inhibitor of the steroid 5-areductase for use in the chemical composition is a synthetic 4-as asteroid compound marketed by Merck & Co., Inc. under the registered trademark Proscar; this active is also called ministered. Finasteride is 4-azaandrost-1-ene-17-carboxamide, N-(1,1-diethyl ethyl)-3-oxo-,(5a,17B)-.

It should be appreciated that duration of treatment according to the method of the chemical composition will vary with many factors and will primarily depend upon the specific condition of the patient, the target situs, and the specific combination of agent employed. It should also be appreciated that both treatment agents, dosage and duration will be interdependent and can be varied tighter in order to achieve an optimal clinical response. In addition, dosage and duration will also depend on the specific combination of agents employed.

The agents utilized in the chemical compositions and method can be administered in accordance with the present invention in any pharmaceutically acceptable carrier, preferably one, which is both non-toxic and suitable for topical or systemic delivery. The compounds may be formulated for administration by procedures well established in the pharmaceutical arts.

For topical administration, pharmaceutically acceptable vehicles in the form or creams, oils, ointments, gels, pastes, liquids, sprays delivery modes known to those skilled in the art may be utilized. A topical vehicle of vehicle component is generally pharmaceutically acceptable if the vehicle of component dose not substantially interfere with the pharmacological activity of the agent or agents and does not cause undue side effects. For example, a pharmaceutical preparation in unit dosage from adapted for administration to promote hair growth may be prepared comprising, per unit dosage at least two active agents selected from the group composition go minoxidil, estradiol and ministered each in a effective non toxic amount within the range of from about 1 to about 300 mg minoxidil; about 1 to about 90 mg estradiol; and about 0.5 to about 15 mg finiastered. Such unit dosage preparations may be administration as liquid, powder, for topical use.

1. What I claim is my invention is to maintain and or increase the eyebrow re-growth by a stimulator for women who has lost their eyebrows due to diseases such as thyroid, cancer, medications, age, hormones. Women are devastated when they loose their eyebrows and the growth pattern that will allow eyebrow pencil other makeup to be applied. The chemical composition of minoxidil 5% and mixtures thereof for promotion of hair re-growth, which is much need in the population of women to re-grow eyebrows?

2. According to claim 1 is an eyebrow re-growth for eyebrows.

1. According to claim 1 is an eyebrow stimulator.

3. According to claim 1 eyebrow hair re-growth is much needed in the population of women.

4. According to claim 1 is the lost eyebrows due to cancer.

5. According to claim 1 is the lost of eyebrows due to age.

6. According to claim 1 is the natural pattern is lost.

7. According to claim 1 is the natural pattern is lost to apply makeup.

8. According to claim 1 is a chemical composition to re-grow eyebrow hairs.

9. According to claim 1 a composition of minoxidil suitable solvent propylene glycol.

10. According to claim 1 woman is devastated when they loose their eyebrows.

11. According to claim 1 minoxidil 5% and mixtures thereof.

12. According to claim 1 the lost of eyebrows due to hormones.

13. According to claim 9 minoxidil is a vasodilator.

14. According to claim 11 estradiols are inhibitor.

15. According to claim 14 5-a-reductase is a steroid inhibitor.

16. According to claim 15 inhibitors pharmaceutically acceptable.

17. According to claim 16 inhibitors and pro-drugs of active agents.

18. According to claim 17 pharmaceutically acceptable salts and esters.

19. According to claim 11 cyproterone acetate DHT exerts its effects by blocking the bindings of DHT dilydroestostrone to its reactors.

20. According to claim 19 finasteriede a pharmaceutically acceptable carrier.

21. According to claim 15 calcium ion influx a calcium blocker such as nifedipine are also contemplated as preferred vasodilators.

22. According to claim 13 is propylene glycol, and water.

23. According to claim 22 in equal parts of ethyl alcohol and propylene solvent.

24. According to claim 23 ethyl alcohol suitable co solvent/penetration enhancers.

25. According to claim 24 it is and/or oleyl alcohol so composition has less oily feel.
